(19) United States
(12) Patent Application Publication
(10) Pub. No.: US 2022/0157432 A1
(43) Pub. Date: May 19, 2022

(54) LACTATION-PERIOD RECIPE RECOMMENDATION METHOD, DEVICE, SYSTEM, APPARATUS AND MEDIUM

(71) Applicant: BOE Technology Group Co., Ltd., Beijing (CN)

(72) Inventors: Xinxin LIU, Beijing (CN); Jiao HUANG, Beijing (CN); Hongliang WANG, Beijing (CN); Yanyang HU, Beijing (CN); Xiaotong TONG, Beijing (CN)

(21) Appl. No.: 17/599,486

(22) PCT Filed: Feb. 25, 2021
(86) PCT No.: PCT/CN2021/077925

§ 371 Date: Sep. 28, 2021

(30) Foreign Application Priority Data
Mar. 6, 2020 (CN) 202010152028.2

(51) Int. Cl. G16H 20/60; G16H 50/70

(52) U.S. Cl. CPC G16H 20/60; G16H 50/70

(57) ABSTRACT

The present application discloses a lactation-period recipe recommendation method, device, system, apparatus and medium. The method includes: receiving a breast milk test result of a breast milk provider; obtaining a daily required energy index of the breast milk provider based on physiological characteristic parameters of the breast milk provider; and then, obtaining a recommended recipe for the breast milk provider based on the breast milk test result and the daily required energy index.

### CROSS-REFERENCE TO RELATED APPLICATION

The present application claims the priority of Chinese patent application No. 202010152028.2, filed on Mar. 6, 2020, which is incorporated herein by reference in its entirety.

### TECHNICAL FIELD

The present disclosure relates to the field of intelligent recommendation technologies, and in particular to a lactation-period recipe recommendation method, device, system, apparatus and medium.

### BACKGROUND

At present, with rapid development of the economy and continuous improvement of people's living standards, more and more attention is paid to dietary health issues, especially dietary nutrition of lactating women is more important. Infancy is the most important early stage of human growth, and nutrients it takes in mainly depend on breast milk. Lactating women's intake of a balanced diet is an important cornerstone to ensure healthy growth of breastfed babies. However, in the past 10 years, a survey result of lactating women in China shows that the lack of nutrients in vegetables, fruits and protein foods in the diet of lactating women leads to the lack or imbalance of nutrients in breast milk, which affects healthy growth of breastfed babies to a certain extent.

### SUMMARY

In one aspect, one embodiment of the present disclosure provides a lactation-period recipe recommendation method, which includes:

receiving a breast milk test result of a breast milk provider;

obtaining a daily required energy index of the breast milk provider based on physiological characteristic parameters of the breast milk provider; and

obtaining a recommended recipe for the breast milk provider based on the breast milk test result and the daily required energy index.

In one aspect, one embodiment of the present disclosure provides a lactation-period recipe recommendation method, which includes:

providing, by a breast milk analyzer, a breast milk test result of a breast milk provider, to a terminal device;

transmitting, by the terminal device, obtained physiological characteristic parameters of the breast milk provider and the breast milk test result to a server;

receiving, by the server, the physiological characteristic parameters of the breast milk provider, and calculating a daily required energy index of the breast milk provider based on the physiological characteristic parameters; and

obtaining, by the server, a recommended recipe for the breast milk provider, based on the breast milk test result and the daily required energy index.

In one aspect, one embodiment of the present disclosure provides a lactation-period recipe recommendation device, which includes:

a receiving unit configured to receive a breast milk test result of a breast milk provider;

an energy obtaining unit configured to obtain a daily required energy index of the breast milk provider based on physiological characteristic parameters of the breast milk provider; and

a recipe obtaining unit configured to obtain a recommended recipe for the breast milk provider based on the breast milk test result and the daily required energy index.

In one aspect, one embodiment of the present disclosure provides a lactation-period recipe recommendation system, which includes:

a breast milk analyzer configured to obtain a breast milk test result of a breast milk provider;

a terminal device configured to receive the breast milk test result of the breast milk provider from the breast milk analyzer, and obtain physiological characteristic parameters of the breast milk provider; and

a server configured to receive, from the terminal device, the physiological characteristic parameters of the breast milk provider and the breast milk test result of the breast milk provider, calculate a daily required energy index of the breast milk provider based on the physiological characteristic parameters, and obtain a recommended recipe for the breast milk provider based on the breast milk test result and the daily required energy index.

In one aspect, one embodiment of the present disclosure provides an electronic device, including: a memory, a processor, and a computer program stored in the memory and executable on the processor, wherein the processor executes the program to implement the method described in the embodiments of the present application.

In one aspect, one embodiment of the present disclosure provides a computer-readable storage medium, including a computer program stored thereon, wherein the computer program is executed by a processor to implement the method described in the embodiments of the present application.

### BRIEF DESCRIPTION OF THE DRAWINGS

By reading detailed description of non-limiting embodiments with reference to the following drawings, other features, purposes and advantages of the present application will become more apparent:

FIG. 1 is a schematic flow chart of a lactation-period recipe recommendation method according to an embodiment of the present application;

FIG. 2 is a schematic flow chart of a step of obtaining a daily required energy index of a breast milk provider according to an embodiment of the present application;

FIG. 3 is a schematic flow chart of a step of obtaining a recommended recipe for a breast milk provider according to an embodiment of the present application;

FIG. 4 is a schematic flow chart of a lactation-period recipe recommendation method according to an embodiment of the present application;

FIG. 5 shows an exemplary structural block diagram of a lactation-period recipe recommendation device according to an embodiment of the present application;

FIG. 6 shows a structural block diagram of a lactation-period recipe recommendation system according to an embodiment of the present application;

FIG. 7 is a schematic flow chart of a lactation-period recipe recommendation method according to an embodiment of the present application; and

FIG. 8 shows a structural block diagram of a computer system suitable for implementing a terminal device or a server according to an embodiment of the present application.

### DETAILED DESCRIPTION

The present application will be further described in detail hereinafter with reference to the drawings and embodiments. It can be understood that the specific embodiments described here are only used to explain the relevant disclosure, but not to limit the disclosure. In addition, it should be noted that, for ease of description, only parts related to the disclosure are shown in the drawings.

It should be noted that the embodiments in the present application and the features in the embodiments may be combined with each other in case that there is no conflict. The present application will be described hereinafter in detail with reference to the drawings and in conjunction with embodiments.

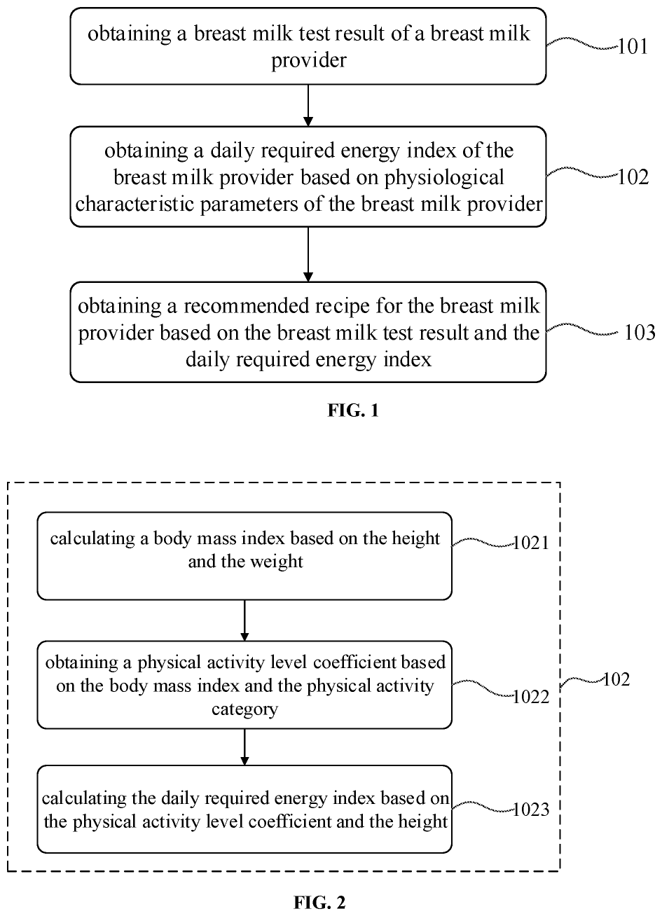

Referring to FIG. 1, FIG. 1 is a schematic flow chart of a lactation-period recipe recommendation method according to an embodiment of the present application.

As shown in FIG. 1, the method includes the following steps.

Step 101: obtaining a breast milk test result of a breast milk provider.

In the foregoing step, the breast milk test result of the breast milk provider may be obtained through a breast milk detector, or the breast milk test result provided by other device may be obtained. The breast milk test result includes but is not limited to fat, protein, lactose, energy, calcium, zinc and other test indicators. The breast milk test result may also be an analysis result of various test indexes obtained by analyzing the baby's month age. For example, each type of test index is classified according to levels, and a level corresponding to each type of test index is obtained by comparing a true value of each type of test index with a standard reference threshold value. The level may be low, normal, or high.

Step 102: obtaining a daily required energy index of the breast milk provider based on physiological characteristic parameters of the breast milk provider.

The physiological characteristic parameters of the breast milk provider include but are not limited to the breast milk provider's height, weight and physical activity level coefficient. The physical activity level coefficient is a pre-defined level of physical exertion based on the labor work performed by the breast milk provider.

The daily required energy index of the breast milk provider may be calculated through the physiological characteristic parameters of the breast milk provider. The daily required energy index is a recommended energy value that the breast milk provider needs to consume on that day in combination with a daily labor amount of the breast milk provider and food-ingredient conversion coefficient.

Step 103: obtaining a recommended recipe for the breast milk provider based on the breast milk test result and the daily required energy index.

In the foregoing step, the obtaining a recommended recipe for the breast milk provider based on the breast milk test result and the daily required energy index of the breast milk provider, includes: obtaining a customized recommended recipe for the breast milk provider from a server, according to evaluation results of various test indexes in the breast milk test result and the daily required energy index of the breast milk provider. The recommended recipe can meet daily energy needs of the breast milk provider on the basis of a balanced combination of ingredients.

In this embodiment of the present application, the breast milk test result and the daily required energy index of the breast milk provider are taken as important influencing factors in the recipe recommendation, thereby providing the breast milk provider with a nutritious, scientific and comprehensive dietary structure, and then effectively overcoming the problem of lack or imbalance of nutrients in breast milk.

Hereinafter, the step 102 is further described in conjunction with the physiological characteristic parameters of the breast milk provider. Referring to FIG. 2, FIG. 2 is a schematic flow chart of the step 102 according to an embodiment of the present application. The physiological characteristic parameters of the breast milk provider include the height, weight and physical activity category of the breast milk provider. As shown in FIG. 2, the step 102 further includes:

Step 1021: calculating a body mass index based on the height and the weight;

Step 1022: obtaining a physical activity level coefficient based on the body mass index and the physical activity category;

Step 1023: calculating the daily required energy index based on the physical activity level coefficient and the height.

In the above steps are performed after obtaining the physiological characteristic parameters of the breast milk provider, such as the height and weight of the breast milk provider, and the physical activity category of the breast milk provider. The physical activity categories are classified according to levels of physical labor performed by the breast milk provider. After calculating the body mass index of the breast milk provider based on the height and weight of the breast milk provider, the physical activity level coefficient can be obtained based on the body mass index of the breast milk provider and the physical activity category of the breast milk provider. Optionally, the physical activity level coefficient may be obtained by looking up a table. As shown in the following table (1), the table (1) shows relationship among the body mass index, the physical activity category and the physical activity level coefficient.

TABLE 1

| physical | body mass | | | |
|---|---|---|---|---|
| activity | index (BMI) | | | |
| category | <18.5 | [18.5,24) | ]24,) | Note |
| Bed rest | 25 | 20 | 15 | lactating women who need bed rest |
| light | 35 | 30 | 25 | office clerk, teacher, salesperson, |
| physical | | | | simple housework, or equivalent |
| labor | | | | amount of activity |
| molderate | 40 | 35 | 30 | students, drivers, surgeons, physical |
| physical | | | | education teachers, general farm |
| labor | | | | work, or equivalent activities |
| heavy | 45 | 40 | 35 | heavy farm work, construction |
| physical | | | | workers, porters, dancers, athletes, or |
| labour | | | | equivalent activities |

As shown in the above table, the body mass index (BMI) of the breast milk provider is calculated according to the height and weight of the breast milk provider with the following formula:

BMI=weight/height square (international unit kg/m²)

After obtaining the body mass index and the physical activity category of the breast milk provider, the physical activity level coefficient of the breast milk provider is obtained by looking up the table. The above activity level coefficient table may be stored in advance in a related database, such as an energy coefficient database.

Then, according to the physical activity level coefficient of the breast milk provider and the height of the breast milk provider, the daily required energy index of the breast milk provider is calculated with the following formula:

the daily required energy index (kcal) of the breast milk provider=(height cm-105)×physical activity level coefficient+500

in this embodiment of the present application, the daily required energy index of the breast milk provider is calculated according to the objectively obtained physiological characteristic parameters of the breast milk provider, and a recommended recipe is individually designed for the breast milk provider, thereby greatly improving the nutritional problem of the breast milk provider.

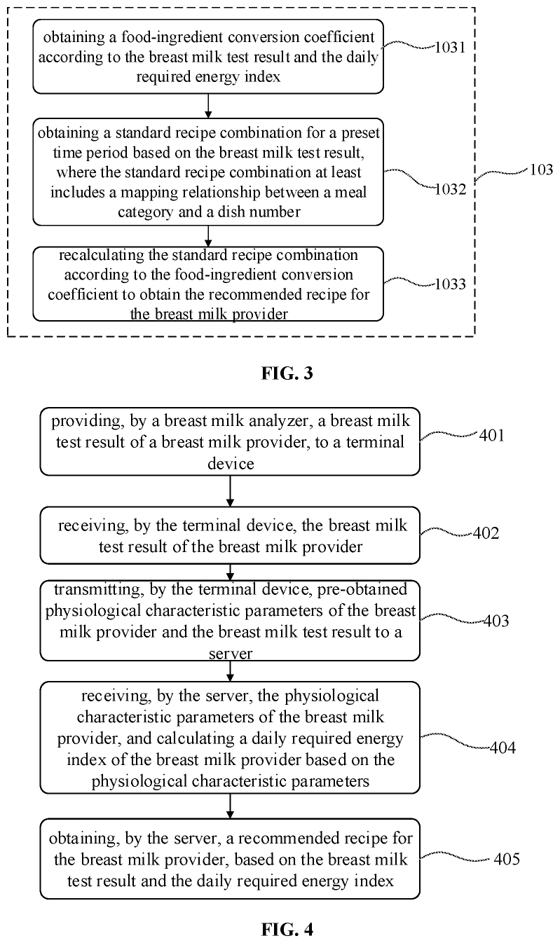

Referring to FIG. 3, FIG. 3 is a schematic flow chart of the step 103 according to an embodiment of the present application. As shown in FIG. 3, the step 103 includes:

Step 1031: obtaining a food-ingredient conversion coefficient according to the breast milk test result and the daily required energy index;

Step 1032: obtaining a standard recipe combination for a preset time period based on the breast milk test result, where the standard recipe combination at least includes a mapping relationship between a meal category and a dish number;

Step 1033: recalculating the standard recipe combination according to the food-ingredient conversion coefficient to obtain the recommended recipe for the breast milk provider.

In the above steps, the breast milk test result includes but is not limited to fat, protein, lactose, energy, zinc, calcium and other test indexes. The breast milk test result may be obtained by comparing the test result with the standard threshold value. For example, in the breast milk test index, a protein index is lower than a standard threshold interval, that is, the protein index is low; a fat index is higher than a standard threshold interval, and then the fat index is high. Optionally, in the present application, the food-ingredient conversion coefficient may be obtained through an energy index in the breast milk test result and the daily required energy index of the breast milk provider. It is assumed that a pre-established energy coefficient table is stored in an energy coefficient database or a corresponding block-chain system. A process of obtaining food-ingredient conversion coefficient is illustrated hereinafter with reference to the following table (2). The table (2) is part of data of a food-ingredient conversion-coefficient table, which is only used to express the scheme and does not limit the content. The food-ingredient conversion coefficient may be understood as an energy coefficient of corresponding food ingredient.

TABLE 2

| | daily required energy index range | | | | | |
|---|---|---|---|---|---|---|
| | (1, 1200) | | | [1200, 1400) | | |
| | breast milk-energy | | | | | |
| coefficient | high | normal | low | high | normal | low |
| grains | 0.31 | 0.31 | 0.38 | 0.38 | 0.50 | 0.62 |
| poultry & meat | 0.20 | 0.20 | 0.33 | 0.33 | 0.40 | 0.50 |
| eggs | 0.40 | 0.40 | 0.50 | 0.50 | 0.50 | 0.50 |

A food-ingredient conversion coefficient of corresponding food may be obtained according to an energy index in the breast milk test result and the daily required energy index of the breast milk provider. It is assumed that the daily required energy index of the breast milk provider is 1300 and the energy index in the breast milk test result is low, then, a food-ingredient conversion coefficient corresponding to grains is 0.38, and a food-ingredient conversion coefficient corresponding to poultry and meat is 0.33.

The recalculating the standard recipe combination according to the food-ingredient conversion coefficient to obtain the recommended recipe for the breast milk provider, may further include:

obtaining a weight value and a unit energy value of an ingredient contained in each dish number according to the dish number in the standard recipe combination;

recalculating the weight value and unit energy value of the ingredient corresponding to each dish number according to the food-ingredient conversion coefficient to obtain an energy value and a weight value related to the recommended recipe.

In the above steps, information related to the recommended recipe includes but is not limited to: the energy value and weight value of the recommended recipe; an analysis report of the recommended recipe; and cooking guidance information of the recommended recipe. The obtaining the recommended recipe for a preset time period according to the breast milk test result, for example, may include obtaining a corresponding recommended recipe according to the breast milk test result. The recommended recipe may include multiple combination relationships. For example, the recommended recipe includes but is not limited to a mapping relationship between a meal category and the dish number. It is assumed that six indexes in the breast milk test result are all low, based on the breast milk test result and the daily required energy index of the breast milk provider, multiple dish numbers are obtained from a recipe recommendation database and are combined, and the combined result is associated with the meal category. Meal categories include but are not limited to breakfast category, lunch category, dinner category, morning snack, afternoon snack, etc. The breakfast category may be selected from a combination of multiple dish numbers. For example, yam porridge is the dish name, zs018 is the dish number, and the corresponding meal category is the breakfast category. For another example, mutton and radish soup is the dish name, jdf057 is the dish number, and the corresponding meal category is the lunch category.

After obtaining the recommended recipe, the weight value and unit energy value of the ingredient corresponding to each dish number are recalculated according to the food-ingredient conversion coefficient to obtain the energy value and weight value related to the recommended recipe.

For example, the food category includes, but is not limited to: grains, potatoes, vegetables, fruits, poultry meat, eggs, aquatic products, dairy products, and soybeans. It is assumed that a recommended dish number is jdf057, ingredients contained in the dish number include mutton and radish, a weight of each type of ingredient is m (unit: grams), a unit energy value of each type of ingredient, and a conversion coefficient of each type of ingredient obtained is λ, then,

a weight value of the dish to be ingested is:

M=m(mutton)*λ(poultry meat)+m(radish)*λ(vegetables)

a corresponding ingestible energy value is:

K=m(mutton)*λ(poultry meat)*k(mutton)+m(radish)*λ(vegetable)*k(radish)

where m (mutton) and m (radish) may be weights predefined in a dish number.

λ(poultry meat), λ(vegetable) are the food-ingredient conversion coefficients of the corresponding ingredients contained in the dish number, λ(poultry meat) may be specific λ(mutton), λ(vegetable) may be specific λ(radish).

k(mutton) and k(radish) are unit energy values corresponding to the ingredients contained in the dish number. The definition of unit energy value refers to an energy value corresponding to 1 gram of each type of food ingredient, or an energy value corresponding to 100 grams of each type of food ingredient.

The weight and energy of the dishes that need to be ingested are a recommended weight and energy values that the breastmilk provider needs to consume after preparing the dishes according to the standard recipe combination. Alternatively, the breast milk provider may complete the preparation of the dishes according to a recommended weight ratio, and consume all the dishes according to the recommended weight ratio.

Optionally, the energy value and weight value may also be summarized according to daily meal situation. For example, a certain dish in a recommended recipe contains A-D ingredients, a weight of each type of ingredient is m(A), m(B), m(C), m(D), the unit energy value is an energy corresponding to 100 grams of each type of ingredient, and a unit energy value corresponding to each type of ingredient is k(A), k(B), k(C), k(D), then a weight value of the dish that needs to be ingested is:

M=m(A)*λ(A)+m(B)*λ(B)+m(C)*λ(C)+m(D)*λ(D)

a corresponding ingestible energy value is:

$$K = \frac{\begin{matrix}m ( A ) * {λ ( A )}^{*} k ( A ) + m ( B ) * {λ ( B )}^{*} k ( B ) + \\ m ( C ) * {λ ( C )}^{*} k ( C ) + m ( D ) * {λ ( D )}^{*} k ( D )\end{matrix}}{100}$$

where λ(A), λ(B), λ(C) and λ(D) are food-ingredient conversion coefficients corresponding to each type of food ingredients, and the food-ingredient conversion coefficients may be obtained by querying a food ingredient database;

m(A), m(B), m(C) and m(D) are weights of each type of ingredients specified in the dish, which may be obtained by querying a recommended recipe database;

k(A), k(B), k(C), k(D) are unit energy values corresponding to ingredients included in the dish number.

In this embodiment of the present application, a recommended recipe for the breast milk provider can be obtained based on the breast milk test result of the breast milk provider and the daily required energy index of the breast milk provider, and the recommended recipe can fully meet energy needs of the breast milk provider and achieve nutritional balance effect.

Optionally, in this embodiment of the present application, a total energy and a total quantity of the recommended recipe may be calculated according to the recommended recipe.

In this embodiment of the present application, the information related to the recommended recipe includes, but is not limited to, the nutritional components, energy value, and weight value of the recipe. The information related to the recommended recipe may further include an analysis report corresponding to the breast milk test result, and cooking guidance information related to the recommended recipe, such as guidance display information of a cooking process of a recommended dish.

After obtaining the recommended recipe for the breast milk provider, the recommended recipe is displayed according to a predefined display template. For example, dish name, dish number and dish nutritional information corresponding to a meal category may be displayed in the display template; an energy value and a weight value of a menu may be displayed under a menu name; an energy value and a weight value corresponding to the meal category may be displayed in a displaying area where the meal category is located. A total energy value and a total weight value of a single day may also be displayed in the display template. There are no restrictions on displayed positions and displayed values. The dish nutritional information may include: contents of carbohydrates, fat, protein, zinc, calcium, etc. per 100 grams of the corresponding dish obtained from a recommendation recipe database.

Further, the recommended recipe may also include a weekly recommended recipe. The weekly recommended recipe includes multiple pages of daily recipe pages. The daily recipe pages may be displayed in chronological order, and recipes for a single day may be displayed separately on each daily recipe page. By operating on the daily recipe page, a user can obtain relevant information of respective recipes, such as operation information of the recipe, an energy value corresponding to the recipe, and analysis information of the recipe. After obtaining the information related to recommended recipes based on the breast milk test result and the daily required energy index, the recommended recipes for one week, including types of meal categories and a recipe number in each meal category included in each day, can be obtained. The recipes recommended here may also be dynamically selectable. For example, the recommended recipe, which is obtained based on the breast milk test result and the daily required energy index, may include multiple alternatives for the user to choose.

The present application further provides a lactation-period recipe recommendation method. Referring to FIG. 4, FIG. 4 is a schematic flow chart of a lactation-period recipe recommendation method according to an embodiment of the present application. As shown in FIG. 4, the method is executed interactively by a breast milk analyzer, a terminal device and a server, and the method includes:

Step 401: providing, by a breast milk analyzer, a breast milk test result of a breast milk provider, to a terminal device;

Step 402: receiving, by the terminal device, the breast milk test result of the breast milk provider;

Step 403: transmitting, by the terminal device, pre-obtained physiological characteristic parameters of the breast milk provider and the breast milk test result to a server;

Step 404: receiving, by the server, the physiological characteristic parameters of the breast milk provider, and calculating a daily required energy index of the breast milk provider based on the physiological characteristic parameters;

Step 405: obtaining, by the server, a recommended recipe for the breast milk provider, based on the breast milk test result and the daily required energy index.

In the above steps, the breast milk of the breast milk provider is analyzed and tested by the breast milk analyzer. For example, the breast milk of the breast milk provider can be analyzed in combination with months of age of the breast-fed baby to obtain the breast milk test result, and the breast milk test result can be provided to the terminal device. Alternatively, original test index data corresponding to the breast milk test result can be provided to the terminal device, and then the terminal device obtains the breast milk test result based on analysis of the original test index data.

The terminal device receives the physiological characteristic parameters of the breast milk provider input in advance by the user, such as height, weight, and physical activity category. After receiving the breast milk test result, the terminal device transmits the breast milk test result and the physiological characteristic parameters of the breast milk provider to the server. The server may be one or more servers, and the server may also be composed of a cloud computing platform and multiple databases.

When the server receives the physiological characteristic parameters of the breast milk provider, the server calculates the daily required energy index of the breast milk provider based on the physiological characteristic parameters. Then, the server obtains a recommended recipe for the breast milk provider from a database based on the breast milk test result and the daily required energy index of the breast milk provider. The server may also obtain a recommended recipe for the breast milk provider from a block-chain system, based on the breast milk test result and the daily required energy index of the breast milk provider.

The server obtains the food-ingredient conversion coefficient according to the breast milk test result and the daily required energy index of the breast milk provider, and converts the energy value of the recommended recipe into a true energy value corresponding to the breast milk provider. Compared with a fixed scalar defined in the related art, in the embodiment of the present application, the energy value provided and displayed to the breast milk provider, is a true value which is calculated based on the breast milk provider's own condition and conforms to the breast milk provider's physiological condition. The recommended recipe, which is provided to the breast milk provider based on the true value, can objectively improve the dietary structure of the breast milk provider, so that breast milk provider can obtain a convenient and quick user experience.

It should be noted that although operations of the method of the present disclosure are described in a specific order in the drawings, this does not require or imply that these operations must be performed in the specific order, or that all the operations shown must be performed to achieve the desired result. Conversely, the steps depicted in the flowchart may change the order of execution. Additionally or alternatively, some steps may be omitted, multiple steps may be combined into one step for execution, and/or one step may be decomposed into multiple steps for execution.

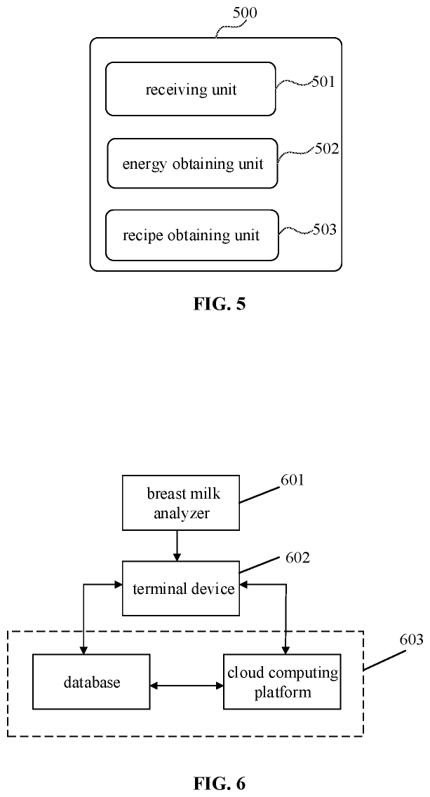

One embodiment of the present application further provides a lactation-period recipe recommendation device. Referring to FIG. 5, FIG. 5 shows an exemplary structural block diagram of a lactation-period recipe recommendation device 500 according to an embodiment of the present application. The lactation-period recipe recommendation device includes:

a receiving unit 501 configured to receive a breast milk test result of a breast milk provider;

an energy obtaining unit 502 configured to obtain a daily required energy index of the breast milk provider based on physiological characteristic parameters of the breast milk provider;

a recipe obtaining unit 503 configured to obtain a recommended recipe for the breast milk provider based on the breast milk test result and the daily required energy index.

The energy obtaining unit 502 further includes:

an index calculation sub-unit configured to calculate a body mass index of the breast milk provider based on the height and the weight of the breast milk provider;

a coefficient obtaining sub-unit configured to obtain a physical activity level coefficient based on the body mass index and the physical activity category of the breast milk provider;

an energy index calculation sub-unit configured to calculate the daily required energy index based on the physical activity level coefficient and the height.

The recipe obtaining unit 503 further includes:

a conversion coefficient obtaining sub-unit configured to obtain food-ingredient conversion coefficient according to the breast milk test result and the daily required energy index;

a standard recipe obtaining sub-unit configured to obtain a standard recipe combination for a preset time period based on the breast milk test result, where the standard recipe combination at least includes a mapping relationship between a meal category and a dish number;

a conversion sub-unit configured to recalculate the standard recipe combination according to the food-ingredient conversion coefficient to obtain the recommended recipe for the breast milk provider.

Optionally, the conversion sub-unit is further configured to,

obtain a weight value and a unit energy value of an ingredient contained in each dish number according to the dish number in the standard recipe combination;

recalculate the weight value and unit energy value of the ingredient corresponding to each dish number according to the food-ingredient conversion coefficient to obtain an energy value and a weight value related to the recommended recipe.

The recipe obtaining unit 503 is further configured to, based on the breast milk test result, obtain an analysis report corresponding to the breast milk test result and cooking guidance information related to the recommended recipe.

The device further includes:

a filling unit configured to fill data corresponding to the recommended recipe into a predefined display template;

a display unit configured to display the recommended recipe in a display mode defined by the display template.

Optionally, when the recommended recipe includes a weekly recommended recipe and the weekly recommended recipe includes multiple pages of daily recipe pages, the display unit is further configured to,

according to timestamps, automatically displaying the daily recipe pages corresponding to the timestamps; or,

in response to an operation instruction input by the user, displaying relevant content corresponding to the operation instruction.

It should be understood that the units or modules recorded in the device 500 are corresponding to the steps in the method described with reference to FIG. 1. Therefore, the operations and features described above with respect to the method are also applicable to the device 500 and the units contained therein, and will not be repeated here. The device 500 may be implemented in a browser or other security application of an electronic device in advance, and may also be loaded into the browser or other security application of the electronic device by downloading. Corresponding units in the device 500 can cooperate with units in the electronic device to implement the solutions of the embodiments of the present application.

For the modules or units mentioned in the foregoing detailed description, such division is not mandatory. In fact, according to the embodiments of the present disclosure, features and functions of two or more modules or units described above may be embodied in one module or unit. Conversely, the features and functions of one module or unit described above may be further divided into multiple modules or units for implementation.

One embodiment of the present application further provides a lactation-period recipe recommendation system. Referring to FIG. 6, FIG. 6 shows a structural block diagram of a lactation-period recipe recommendation system according to an embodiment of the present application. As shown in FIG. 6, the system includes:

a breast milk analyzer 601 configured to obtain a breast milk test result of a breast milk provider and provide the breast milk test result to a terminal device;

the terminal device 602 configured to receive the breast milk test result of the breast milk provider, and transmit pre-obtained physiological characteristic parameters of the breast milk provider and the breast milk test result to a server;

the server 603 configured to receive the physiological characteristic parameters of the breast milk provider, calculate a daily required energy index of the breast milk provider based on the physiological characteristic parameters, and obtain a recommended recipe for the breast milk provider based on the breast milk test result and the daily required energy index.

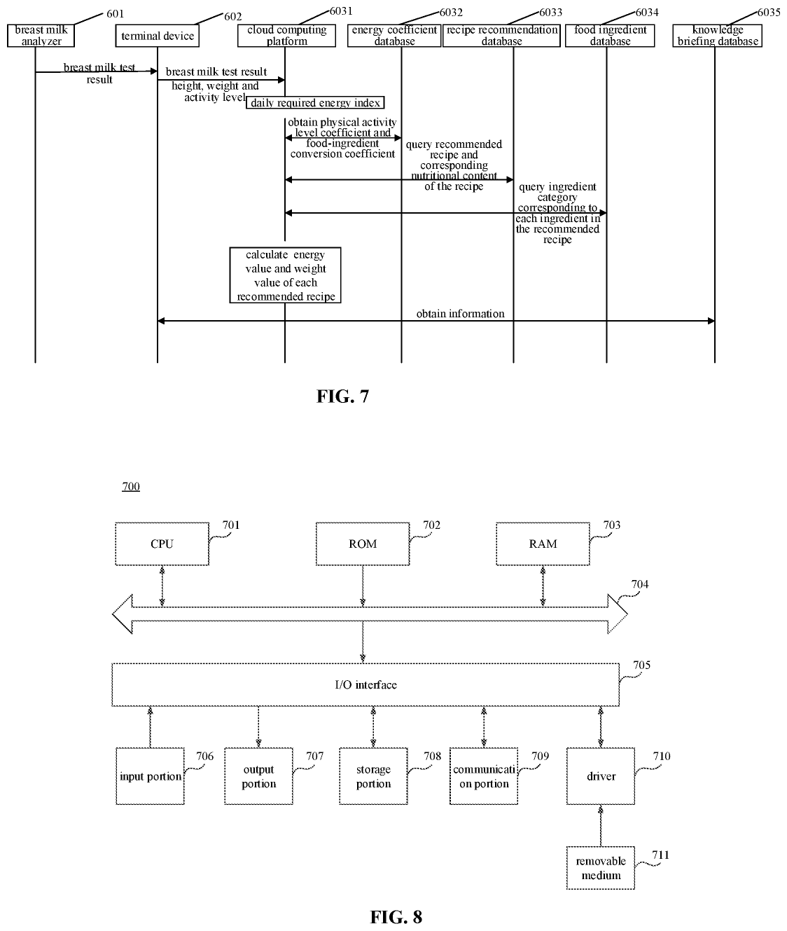

As shown in FIG. 6 and FIG. 7, the server 603 may include a cloud computing platform 6031 and multiple databases, such as an energy coefficient database 6032, a recommendation recipe database 6033, a food ingredient database 6034, and a knowledge briefing database 6035. The cloud computing platform 6031 and the terminal device 602 are connected wirelessly or wired. A working principle of the lactation-period recipe recommendation method proposed in the embodiment of the present application will be further described hereinafter in conjunction with FIG. 7. FIG. 7 is a schematic flow chart of a lactation-period recipe recommendation method according to an embodiment of the present application.

The terminal device 602 receives a breast milk test result from the breast milk analyzer 601.

The terminal device 602 receives physiological characteristic parameters input by the user.

The terminal device 602 transmits the breast milk test result and the physiological characteristic parameters of the breast milk provider to the cloud computing platform 6031.

The cloud computing platform 6031 calculates a daily required energy index of the breast milk provider based on the obtained physiological characteristic parameters. The daily required energy index is a daily energy demand of the breast milk provider.

When calculating the daily required energy index of the breast milk provider, the cloud computing platform 6031 needs to query the energy coefficient database 6032 to obtain a physical activity level coefficient and calculate the daily required energy index of the breast milk provider by combining the obtained physical activity level coefficient with the above-described calculation of the daily required energy index.

The cloud computing platform 6031 queries, according to the breast milk test result, the recipe recommendation database 6033 to obtain a standard recipe combination for a preset time period. For example, one week's standard recipe combination may be obtained.

The cloud computing platform 6031 recalculates the obtained standard recipe combination according to the food-ingredient conversion coefficient to obtain the recommended recipe for the breast milk provider. For example, the standard recipe combination includes yam porridge for breakfast. The yam porridge contains 100 grams of white rice, 100 grams of white sugar, 100 grams of yam, etc. The food-ingredient conversion coefficients, which are obtained according to an energy index in the breast milk test result of the breast milk provider, are 0.38 for grains, 0.27 for yam, and 0.13 for white sugar. Then, a recalculated result is that 38 grams of white rice, 27 grams of yam and 13 grams of white sugar are needed. The corresponding energy value is also recalculated according to the corresponding conversion coefficients.

The cloud computing platform 6031 returns the calculation result and the recommended recipe to the terminal device 602. The terminal device displays the recommended recipe and the calculation result according to a display template of a pre-installed application.

The ways for the terminal device to display the recommended recipe include but are not limited to one or more of the following combinations: graphic format, audio format, video format, small video, small card video, etc.

The displayed content may include, but is not limited to, the breast milk test result, an analysis report of the breast milk test result, popular science content related to the recommended recipe, a daily recipe page, a recipe detail page corresponding to the dish number included in the recommended recipe, and so on. The popular science content may be obtained by the terminal device 602 from the knowledge briefing database 6035. The recipe detail page shows in detail cooking steps of the recipe.

The format of the display template may be, for example, HTML5 format.

The lactation-period recipe recommendation method provided in the embodiments of the present application combines the breast milk test result and the physiological characteristic parameters of the breast milk provider to provide the breast milk provider with a targeted and personalized recommended recipe, which helps to improve the dietary structure of the breast milk provider, meet needs of reasonable diet while ensuring needs of the breast milk provider for various physiological activities. On the basis of the above-mentioned embodiments, the embodiments of the present application further proposes that the recommended recipe and information related to the recommended recipe may be displayed through a visual and operable interface, so as to provide users of products with convenient and quick operation methods and increase users' experience of using the products.

Referring to FIG. 8, FIG. 8 shows a structural block diagram of a computer system 700 suitable for implementing a terminal device or a server according to an embodiment of the present application.

As shown in FIG. 8 the computer system 700 includes a central processing unit (CPU) 701, which may execute various appropriate actions and processes in accordance with a program stored in a read-only memory (ROM) 702 or a program loaded into a random access memory (RAM) 703 from a storage portion 708. The RAM 703 also stores various programs and data required by operations of the system 700. The CPU 701, the ROM 702 and the RAM 703 are connected to each other through a bus 704. An input/output (I/O) interface 705 is also connected to the bus 704.

The following components are connected to the I/O interface 705: an input portion 706 including a keyboard, a mouse etc.; an output portion 707 including a cathode ray tube (CRT), a liquid crystal display device (LCD), a speaker etc.; a storage portion 708 including a hard disk and the like; and a communication portion 709 including a network interface card, such as a LAN card and a modem. The communication portion 709 performs communication processes via a network, such as the Internet. A driver 710 is also connected to the I/O interface 705 as required. A removable medium 711, such as a magnetic disk, an optical disk, a magneto-optical disk, and a semiconductor memory, may be installed on the driver 710, to facilitate the retrieval of a computer program from the removable medium 711, and the installation thereof on the storage portion 708 as needed.

In particular, according to an embodiment of the present disclosure, the process described above with reference to the flow chart shown in FIG. 1 may be implemented in a computer software program. For example, an embodiment of the present disclosure includes a computer program product, which includes a computer program that is tangibly embedded in a machine-readable medium. The computer program includes program codes for executing the method as illustrated in the flow chart. In such an embodiment, the computer program may be downloaded and installed from a network via the communication portion 709, and/or may be installed from the removable media 711. The computer program, when executed by the CPU 701, implements the functions as defined by the methods of the present disclosure.

It should be noted that the computer-readable medium shown in the present disclosure may be a computer-readable signal medium or a computer-readable storage medium, or any combination of the two. The computer-readable storage medium may be, for example, but not limited to, an electronic, magnetic, optical, electromagnetic, infrared, or semiconductor system, apparatus, or device, or any combination of the above. More specific examples of the computer readable storage medium include but not limited to: an electrical connection with one or more wires, portable computer disk, hard disk, random access memory (RAM), read only memory (ROM), erasable programmable read only memory (EPROM or flash), optical fiber, portable compact disk read only memory (CD-ROM), optical storage device, magnetic storage device, or any suitable combination of the above. In some embodiments of the present disclosure, the computer-readable storage medium may be any tangible medium that contains or stores a program, and the program may be used by or used in connection with a command execution system, apparatus, or device. The computer-readable signal medium may include data signal in the base band or propagating as parts of a carrier, in which computer readable program codes are carried. The propagating data signal may take various forms, including but not limited to: an electromagnetic signal, an optical signal or any suitable combination of the above. The signal medium that can be read by computer may be any computer-readable medium except for the computer readable storage medium. The computer-readable medium is capable of transmitting, propagating or transferring programs for use by, or used in combination with, a command execution system, apparatus or element. The program codes embodied in the computer-readable medium can be transmitted by any suitable medium, including but not limited to: wireless, wire, optic cable, radio frequency (RF), etc., or any suitable combination of the foregoing.

The flowcharts and block diagrams in the figures illustrate architectures, functions and operations that may be implemented according to the system, the method and the computer program product of the various embodiments of the present disclosure. In this regard, each block in the flowcharts and block diagrams may represent a module, a program segment, or a code portion. The module, the program segment, or the code portion includes one or more executable instructions for implementing the specified logical function. It should be noted that, in some alternative implementations, the functions denoted by the blocks may occur in a sequence different from the sequences shown in the figures. For example, in practice, two blocks in succession may be executed, depending on the involved functionalities, substantially in parallel, or in a reverse sequence. It should also be noted that, each block in the block diagrams and/or the flow charts and/or a combination of the blocks may be implemented by a dedicated hardware-based system executing specific functions or operations, or by a combination of a dedicated hardware and computer instructions.

The described units or modules involved in the embodiments of the present application may be implemented in software or hardware. The described unit or module may also be provided in the processor, for example, it may be described as: a processor includes a receiving unit, an energy obtaining unit, and a recipe obtaining unit. Names of these units or modules do not constitute a limitation on the units or modules themselves under certain circumstances. For example, the receiving unit may also be described as “a unit for receiving a breast milk test result from a breast milk provider”.

In another aspect, the present application further provides a computer-readable storage medium. The computer-readable storage medium may be a computer-readable storage medium included in the electronic device described in the foregoing embodiment, or a stand-alone computer-readable storage medium which has not been assembled into the electronic device. The computer-readable storage medium stores one or more programs. The one or more programs, when executed by one or more processors, cause the one or more processors to implement the lactation-period recipe recommendation method of the present application.

The foregoing is only a description of the preferred embodiments of the present application and the applied technical principles. It should be appreciated by those skilled in the art that the inventive scope of the present application is not limited to the technical solutions formed by the particular combinations of the above technical features. The inventive scope should also cover other technical solutions formed by any combinations of the above technical features or equivalent features thereof without departing from the concept of the invention, such as, technical solutions formed by replacing the features as disclosed in the present application with (but not limited to), technical features with similar functions.

1. A lactation-period recipe recommendation method, comprising:
receiving a breast milk test result of a breast milk provider;
obtaining a daily required energy index of the breast milk provider based on physiological characteristic parameters of the breast milk provider; and
obtaining a recommended recipe for the breast milk provider based on the breast milk test result and the daily required energy index.

2. The method according to claim 1, wherein the physiological characteristic parameters includes: a height, a weight and a physical activity category of the breast milk provider; the obtaining a daily required energy index of the breast milk provider based on physiological characteristic parameters of the breast milk provider, includes:
calculating a body mass index based on the height and the weight;
obtaining a physical activity level coefficient based on the body mass index and the physical activity category; and
calculating the daily required energy index based on the physical activity level coefficient and the height.

3. The method according to claim 1, wherein the obtaining a recommended recipe for the breast milk provider based on the breast milk test result and the daily required energy index, includes:
obtaining a food-ingredient conversion coefficient according to the breast milk test result and the daily required energy index;
obtaining a standard recipe combination for a preset time period based on the breast milk test result, wherein the standard recipe combination at least includes a mapping relationship between a meal category and a dish number; and
recalculating the standard recipe combination according to the food-ingredient conversion coefficient to obtain the recommended recipe for the breast milk provider.

4. The method according to claim 3, wherein the recalculating the standard recipe combination according to the food-ingredient conversion coefficient to obtain the recommended recipe for the breast milk provider, includes:
according to dish numbers in the standard recipe combination, obtaining a weight value and a unit energy value of an ingredient contained in each dish number;
recalculating the weight value and unit energy value of the ingredient corresponding to each dish number according to the food-ingredient conversion coefficient to obtain an energy value and a weight value related to the recommended recipe.

5. The method according to claim 1, wherein the obtaining a recommended recipe for the breast milk provider based on the breast milk test result and the daily required energy index, further includes: based on the breast milk test result, obtaining an analysis report corresponding to the breast milk test result and cooking guidance information related to the recommended recipe.

6. The method according to claim 1, wherein after the obtaining a recommended recipe for the breast milk provider based on the breast milk test result and the daily required energy index, the method further includes:
filling data corresponding to the recommended recipe into a predefined display template; and
displaying the recommended recipe in a display mode defined by the display template.

7. The method according to claim 6, wherein the recommended recipe includes a weekly recommended recipe, and the weekly recommended recipe includes multiple pages of daily recipe pages; the displaying the recommended recipe in a display mode defined by the display template, includes:
according to timestamps, automatically displaying the daily recipe pages corresponding to the timestamps; or,
in response to an operation instruction input by a user, displaying relevant content corresponding to the operation instruction.

8. The method according to claim 6, wherein the displaying the recommended recipe in a display mode defined by the display template, includes:
displaying the recommended recipe in at least one of a graphic format, an audio format, and a video format.

9. The method according to claim 8, wherein the displaying the recommended recipe in at least one of a graphic format, an audio format, and a video format, includes:
displaying the recommended recipe on a display device in at least one of a graphic format, an audio format, and a video format.

10. A lactation-period recipe recommendation method, comprising:
providing, by a breast milk analyzer, a breast milk test result of a breast milk provider, to a terminal device;
transmitting, by the terminal device, obtained physiological characteristic parameters of the breast milk provider and the breast milk test result to a server;
receiving, by the server, the physiological characteristic parameters of the breast milk provider, and calculating a daily required energy index of the breast milk provider based on the physiological characteristic parameters; and
obtaining, by the server, a recommended recipe for the breast milk provider, based on the breast milk test result and the daily required energy index.

11-12. (canceled)

13. An electronic device, comprising: a memory, a processor, and a computer program stored in the memory and executable on the processor, wherein the processor executes the program to implement:
receiving a breast milk test result of a breast milk provider;
obtaining a daily required energy index of the breast milk provider based on physiological characteristic parameters of the breast milk provider; and
obtaining a recommended recipe for the breast milk provider based on the breast milk test result and the daily required energy index.

14. A computer-readable storage medium, comprising a computer program stored thereon, wherein the computer program is executed by a processor to implement the method according to claim 1.

15. The device according to claim 13, wherein the physiological characteristic parameters includes: a height, a weight and a physical activity category of the breast milk provider; the processor executes the program to implement:
calculating a body mass index based on the height and the weight;
obtaining a physical activity level coefficient based on the body mass index and the physical activity category; and
calculating the daily required energy index based on the physical activity level coefficient and the height.

16. The device according to claim 13, wherein the processor executes the program to implement:
obtaining a food-ingredient conversion coefficient according to the breast milk test result and the daily required energy index;
obtaining a standard recipe combination for a preset time period based on the breast milk test result, wherein the standard recipe combination at least includes a mapping relationship between a meal category and a dish number; and
recalculating the standard recipe combination according to the food-ingredient conversion coefficient to obtain the recommended recipe for the breast milk provider.

17. The device according to claim 16, wherein the processor executes the program to implement:
according to dish numbers in the standard recipe combination, obtaining a weight value and a unit energy value of an ingredient contained in each dish number;
recalculating the weight value and unit energy value of the ingredient corresponding to each dish number according to the food-ingredient conversion coefficient to obtain an energy value and a weight value related to the recommended recipe.

18. The device according to claim 13, wherein the processor executes the program to implement: based on the breast milk test result, obtaining an analysis report corresponding to the breast milk test result and cooking guidance information related to the recommended recipe.

19. The device according to claim 13, wherein after the obtaining a recommended recipe for the breast milk provider based on the breast milk test result and the daily required energy index, the processor executes the program to implement:
filling data corresponding to the recommended recipe into a predefined display template; and
displaying the recommended recipe in a display mode defined by the display template.

20. The device according to claim 19, wherein the recommended recipe includes a weekly recommended recipe, and the weekly recommended recipe includes multiple pages of daily recipe pages; the processor executes the program to implement:
according to timestamps, automatically displaying the daily recipe pages corresponding to the timestamps; or,
in response to an operation instruction input by a user, displaying relevant content corresponding to the operation instruction.

21. The device according to claim 19, wherein the processor executes the program to implement:
displaying the recommended recipe in at least one of a graphic format, an audio format, and a video format.

22. The device according to claim 21, wherein the processor executes the program to implement:
controlling a display device to display the recommended recipe in at least one of a graphic format, an audio format, and a video format.
